(19) United States
(12) Patent Application Publication
(10) Pub. No.: US 2012/0184928 A1
(43) Pub. Date: Jul. 19, 2012

(54) STEP N SOAK SYSTEMS

(76) Inventors: Lydia DelRossi, Mississauga (CA); Ryerson Riley, Aiken, SC (US)

(21) Appl. No.: 13/116,624

(22) Filed: May 26, 2011

Related U.S. Application Data
(60) Provisional application No. 61/433,802, filed on Jan. 18, 2011.

(51) Int. Cl. A61F 13/06; A61M 35/00

(52) U.S. Cl. 604/293

(57) ABSTRACT

An appendage soaking system comprising at least one flexible non-permeable container and at least one securing assembly. The flexible non-permeable container comprises a plastic bag. The securing assembly may be a fused strap(s) oriented parallel to the bottom edge and in close proximity to the open top so that the fused strap may be tied around the appendage. The plastic bag comprises a medicating boot with at least one medicating solution that is able to be suitably contained within the flexible container. Appendages may be inserted into the flexible container which may include a foot, a hoof, or a paw. The strap is secured around the appendage thereby defining a confined inner volume wherein the medicating fluid is able to contact the appendage creating a medicating action. The medicating boot is portable such that a user can move about while medicating the appendage.

### CROSS-REFERENCE TO RELATED APPLICATION

The present application is related to and claims priority from prior provisional application Ser. No. 61/433,802, filed Jan. 18, 2011 which application is incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

1. Field of the Invention

The present invention relates generally to the field of soaking devices and more specifically relates to soaking devices for medicating appendages.

2. Description of the Related Art

In modern society, many spas and salons thrive in part because of the amount of people that want relief from their aching feet. The majority of the working world spends at least six and one half hours on their feet every day and by the end of a work week, many find pain relief in their feet from soaking their feet in warm water. Much of this is done in salons where the individual can relax and unwind. Due to the significant amount of time it takes a person to be immobile, soaking of one's feet is not done as often as one would like. Further, the cost of foot soaking services becomes pricey at these salons and spas. In hot, dry climates, the skin on a person's foot often has a tendency to be very dry and calloused, even to the point of cracking around the bottom of the heels, balls of the feet, and toes. This dry cracking condition can be severe and is often very painful. A common way to relive and alleviate dry cracked feet is to medicate the dry, cracked area.

Other conditions of the feet that happen frequently require some kind of treatment as well. Athlete's foot (also known as Ringworm of the Foot, Tinea Pedis, or Hong Kong foot), is a fungal infection of the skin that causes scaling, flaking, and itch of affected areas. It is caused by fungi in the genus Trichophyton and is typically transmitted in moist areas where people walk barefoot, such as in showers or bathhouses. Although the condition typically affects the feet, it can spread to other areas of the body, including the groin. Athlete's foot can be treated by a number of pharmaceutical creams, ointments, sprays and other treatments; however care must be taken to isolate the feet from contact with walking surfaces to maximize the effect of the treatment and to avoid additional infection. Soaking the feet in a medicated fluid is an effective treatment used to treat Athlete's foot.

Filling an open container with a medicated fluid large enough to insert the feet into may prove costly depending on the type of medication used, along with much of the fluid going to waste having never contacted the skin. When a gel, paste, or cream is used, the feet have to be covered in such a way as to not absorb the medication away from the skin. Absorption into the skin by a medication in needed to reach subsurface bacteria or fungi.

Similar conditions can exist for animals as well. Hoof care is especially important when caring for horses because they can experience a severe cracking of the hoof due to dryness and for various other reasons. Although many horses are quite healthy without daily care, lack of hoof-care may pose several problems, which if left unattended, can result in short or long-term health issues for the horse. Hoof dressing is a liquid substance that may be used on the hooves to improve their moisture content, which in turn helps prevent hoof cracks, lost shoes, tender feet, and other common hoof problems. Soaking of the hoof and other treatments with a medicated fluid can greatly improve the appearance of the hooves in show horses.

Canines can experience cracking of the paws that may go deep enough to cause pain or cause nail infections which may create paw irritation, swelling, abnormal nail color and/or nail deformity. Wet or moist areas in soil can harbor fungi that may attack a canine's paws and nail areas. Applying a protective bootie to protect the area to keep any bacteria or fungus from spreading to other areas of the body is important, especially if the dog is in the house much of the time since the fungus can be transmitted to human feet. Complete tissue contact for durations of time with saline solutions or medicated fluids will usually eliminate the problem. Other treatments can include gels or creams. Treatment of animals is not always easy due to difficulties in restricting their movement.

Various attempts have been made to solve the above-mentioned problems such as those found in U.S. Pat. and Pub. Nos. 3,324,580; 2008/0015525; 2004/0147616; 4,036,220; D267,832; and 4,911,151. This prior art is representative of appendage medicating containers. None of the above inventions and patents, taken either singly or in combination, is seen to describe the invention as claimed.

Ideally, a StepNSoak system should be versatile and convenient in-use, yet would operate reliably and be manufactured at a modest expense. Thus, a need exists for a reliable StepNSoak system and to avoid the above-mentioned problems.

### BRIEF SUMMARY OF THE INVENTION

In view of the foregoing disadvantages inherent in the known appendage soaking system art, the present invention provides a novel StepNSoak system. The general purpose of the present invention, which will be described subsequently in greater detail, is to provide greater mobility while soaking appendages thereby providing greater convenience.

The present invention, Step N Soak systems, as disclosed herein preferably comprises: at least one flexible non-permeable container and at least one securing assembly. The flexible non-permeable container preferably comprises a plastic or vinyl bag with at least two seamed side edges, at least one seamed bottom edge, and at least one open top. The plastic or vinyl bag may be comprised of heavy gauge material so that the flexible non-permeable container is reusable. The container has at least one securing assembly which may be a fused strap wherein at least one fused strap is preferably oriented parallel to the seamed bottom edge and the open top (for ease of opening/closing.)

The fused strap is preferably located in close proximity to the open top such that the fused strap may be tied around the appendage at that end of the bag. The flexible non-permeable container may comprise exactly two fused straps that may be criss-crossed (or otherwise laced/tied/orientated) around the appendage, or the flexible non-permeable container may comprise exactly one fused strap which can be wrapped around the circumference of the appendage and tied. The strap may be fused in the middle of the strap to one side of the flexible non-permeable container to provide two loose ends for tying the bag closed or one strap fused at one end so that fused strap is made integral with the flexible non-permeable container using a heat stamping process.

The bag may comprise a medicating boot with at least one medicating solution that is able to be suitably contained within the flexible container which may be a saline solution or other suitable/appropriate medicating fluid. At least one appendage is able to be inserted into the flexible container which may be a foot for use with humans, a hoof for use with horses, or a paw for use with canines (as examples but not limited to) with the strap being secured around the appendage thereby defining a confined inner volume wherein the medicating fluid is able to contact the appendage creating one medicating action. The medicating boot is portable such that a user can move about (freely) while yet medicating the appendage.

The design currently may be used as a “hoof-foot-paw” soaking boot/bag with a design which can be about 14″ wide—about 21″ tall has a single about 42″ long by about 1″ wide single side tie secured 3″ down from the top which criss-crosses around the leg to secure and/or can be made with (2 of) about 20″ long by about 1″ wide ties secured on either side. These ties can crisscross around the leg to secure and/or may be made with Velcro ties which repeat the same overall construction design pattern options but use Velcro ties to removably attach. The product may be manufactured with the same basic design in smaller dimensions for example the dog/cat Veterinarian paw product will be a much smaller version.

StepNSoak is multi-dimensional and can be folded in half to become only about 7″ wide and rolled down to fit a child's foot—a small hoof and/or zoo animal paw and may comprise “soft matte vinyl” construction or alternately may be constructed in a variety of materials such as vinyl, rubber, canvas, or rubberized cloth (or other suitable equivalent material(s)). The present invention may also be used for foot—toe ankle Rx application and lower leg/foot icing of fractures, sprains and swelling.

The appendage soaking system may further comprise a kit including a plurality of medicating boots; at least one medicating fluid; and a set of user instructions.

In accordance with the embodiments of the present invention a preferred method of use is disclosed herein comprising: opening one flexible non-permeable container; inserting one appendage; pouring one medicated fluid into the flexible non-permeable container; wearing the flexible non-permeable container creating one medicating action; removing the flexible non-permeable container; cleaning the flexible non-permeable container; and storing the flexible non-permeable container.

The present invention holds significant improvements and serves as an appendage soaking system. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, StepNSoak systems, constructed and operative according to the teachings of the present invention.

FIG. 1 shows StepNSoak systems in an ‘in-use’ condition on humans according to an embodiment of the present invention.

FIG. 2 shows StepNSoak systems in an ‘in-use’ condition on horses according to an embodiment of the present invention of FIG. 1. wherein flexible non-permeable container comprises a plastic bag.

FIG. 3 is a perspective view illustrating the StepNSoak systems according to an embodiment of the present invention of FIG. 1.

FIG. 4 is another perspective view illustrating the StepNSoak systems according to an embodiment of the present invention of FIG. 1.

FIG. 5 is a flowchart illustrating a method of use of StepNsoak systems according to an embodiment of the present invention of FIGS. 1-4.

The various embodiments of the present invention will hereinafter be described in conjunction with the appended drawings, wherein like designations denote like elements.

### DETAILED DESCRIPTION

As discussed above, embodiments of the present invention relate to a soaking device and more particularly to a soaking device for medicating appendages as used to improve the convenience and cost for medicating appendages.

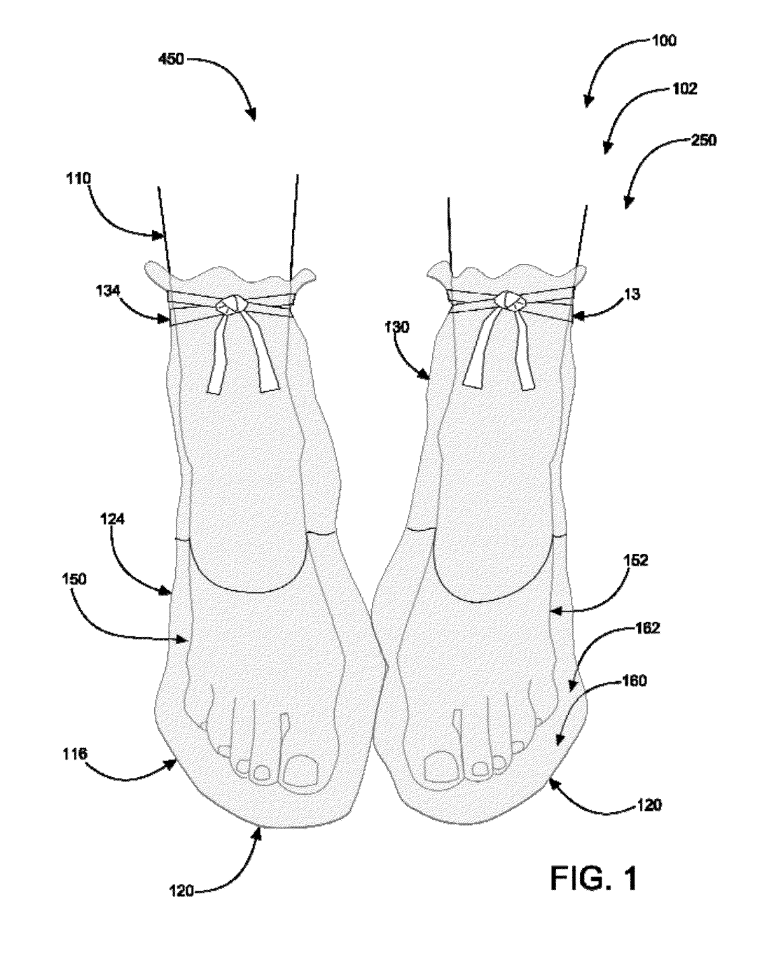

Referring now to the drawings by numerals of reference there is shown in FIG. 1, StepNSoak Systems 100 in ‘an in-use’ condition on human(s) 110 according to an embodiment of the present invention. StepNSoak Systems 100 preferably comprises appendage soaking system 102 including at least one flexible non-permeable container 120 and at least one securing assembly 130. At least one medicating solution 140 is able to be suitably contained within flexible non-permeable container 120 wherein at least one appendage 150 is able to be inserted into flexible non-permeable container 120.

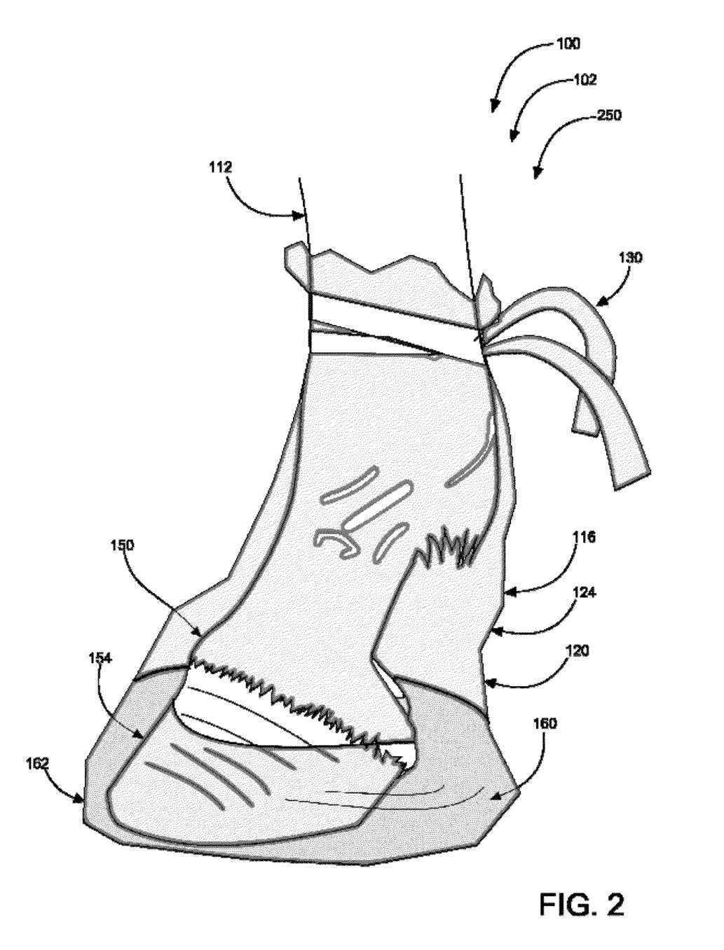

Appendage 150 may comprise foot 152 of a human, however hoof 154 of a horse or a paw of an animal such as a cat or dog may be suitable (as shown in FIG. 2). Securing assembly 130 is preferably secured around appendage 150 thereby defining a confined inner volume wherein medicating fluid 160 is able to contact appendage 150 thereby creating at least one medicating action. Medicating fluid 160 may comprise saline solution 162 or may comprise a user defined medication. Medicating boot 124 preferably comprises heavy gauge vinyl or flexible plastic and is portable such that a user can move about while medicating appendage 150.

Referring now to FIG. 2, showing StepNSoak Systems 100 in another ‘in-use’ condition 250 on horse(s) 112 according to an embodiment of the present invention of FIG. 1. StepNSoak Systems 100 may be used for medicating appendage(s) 150 of most larger animals/pets in a like manner.

StepNSoak Systems 100 may be used for equine purposes wherein appendage(s) 150 may comprise hoof 154 or for canine purposes where appendage(s) 150 may comprise a paw (bigger or smaller bag(s) 116 may be used accordingly.) Bag 116 comprises medicating boot 124 that may be partially filled with medicating fluid 160. Medicating fluid 160 may comprise saline solution 162, however other medicating fluids may be alternately used (or in conjunction with saline solution 162.) Medicating fluid 160 when used in conjunction with flexible non-permeable container 120 is preferably portable such that a user can move about while medicating appendage(s) 150.

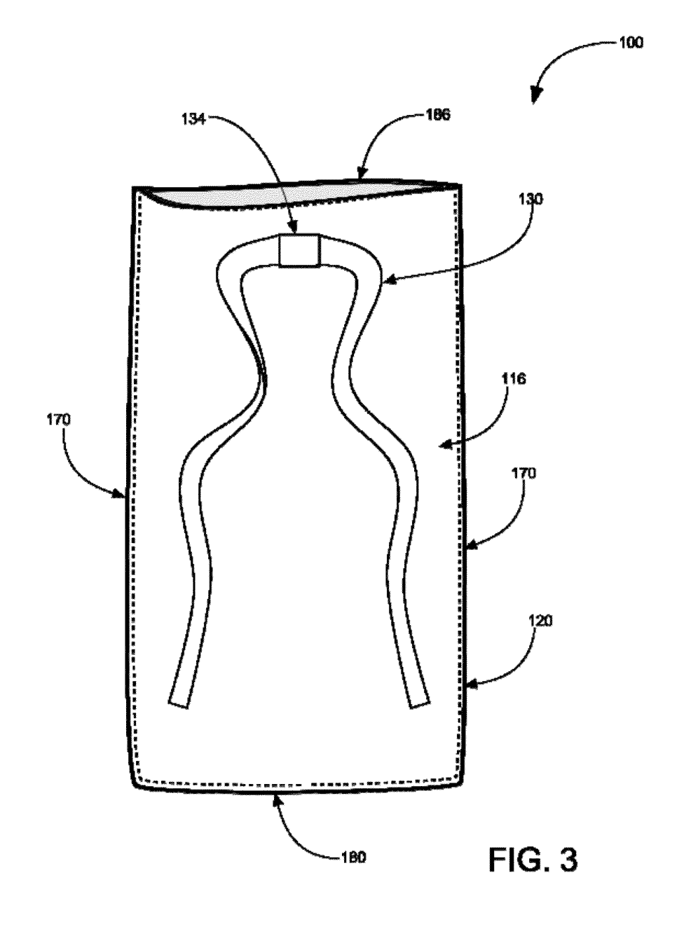

Referring now to FIG. 3, showing a perspective view illustrating StepNSoak Systems 100 according to an embodiment of the present invention of FIG. 1. Bag 116 is preferably manufactured from plastic or vinyl and may preferably comprise two seamed side edge(s) 170, seamed bottom edge 180, and open top 186. (Other embodiments may comprise more or less sides, openings, edges.) Bag 116 preferably is comprised of heavy gauge material such that flexible non-permeable container 120 is reusable.

At least one securing assembly 130 may be included for tying around appendage(s) 150 to suitably contain at least one medicating fluid 160. The securing assembly 130 (fused strap 134) is preferably oriented parallel to seamed bottom edge 180 and open top 186, as shown. At least one fused strap 134 is located in close proximity to open top 186 but flexible non-permeable container 120 may comprise exactly two fused strap(s) 134 in some embodiments. Fused strap 134 is made integral with flexible non-permeable container 120 via a heated stamp(ing process.)

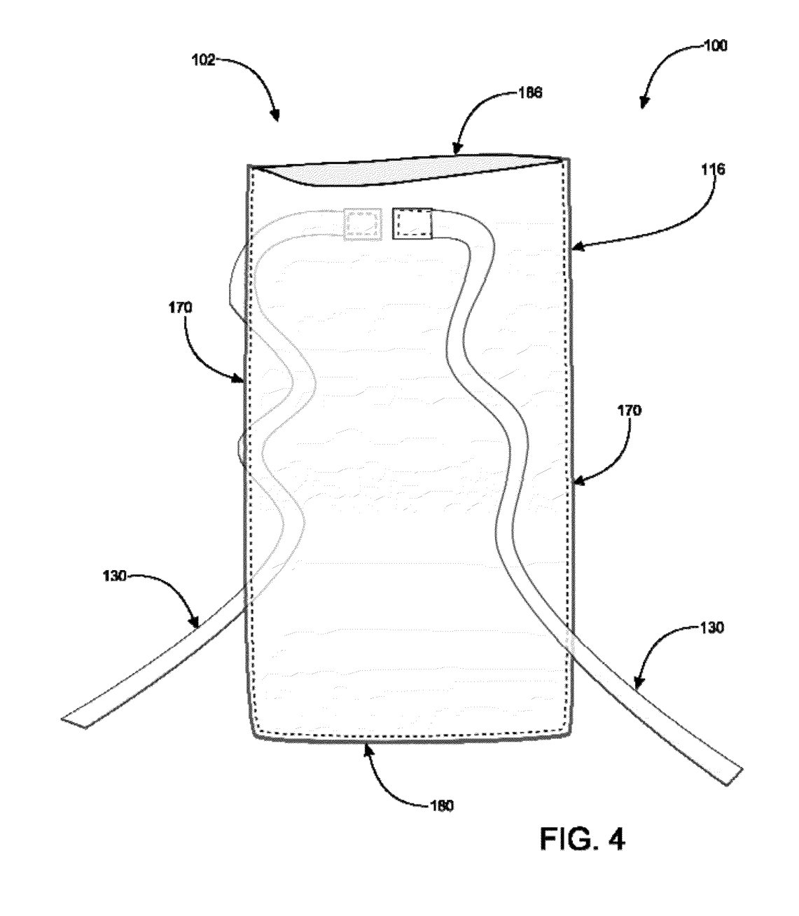

Referring now to FIG. 4, showing yet another perspective view illustrating

StepNSoak Systems 100 according to an embodiment of the present invention of FIG. 1. Flexible non-permeable container 120 may also comprise exactly one fused strap 134 but in other embodiments may comprise exactly two fused strap(s) 134. When exactly one fused strap 134 is used, fused strap 134 is preferably fused in the middle and preferably to one side of flexible non-permeable container 120 such that fused strap 134 is made integral with flexible non-permeable container 120. Fused strap 134 is made integral with flexible non-permeable container 120 via a heated stamping process as mentioned previously. It should be appreciated that other manufacturing processes may be used and that the present invention may be used for a variety of medicating applications not limited to feet, legs, hoofs, paws, hand, but rather to a wide variety of applications wherein the containment means may be used (and re-used.)

StepNSoak Systems 100 may be sold as kit 450 comprising the following parts: a plurality of medicating boot(s) 124; at least one medicating fluid 160; and at least one set of user instructions. StepNSoak Systems 100 may be manufactured and provided for sale in a wide variety of sizes and shapes for a wide assortment of applications. Upon reading this specification, it should be appreciated that, under appropriate circumstances, considering such issues as design preference, user preferences, marketing preferences, cost, structural requirements, available materials, technological advances, etc., other kit contents or arrangements such as, for example, including more or less components, customized parts, different color combinations, parts may be sold separately, etc., may be sufficient.

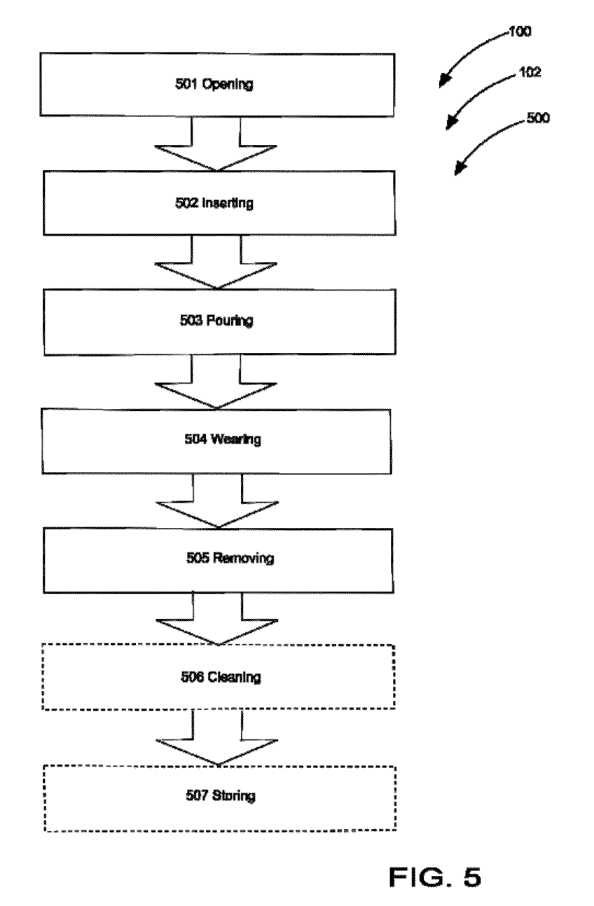

Referring now to FIG. 5, a chart showing a method of use for StepNSoak Systems 100 according to an embodiment of the present invention of FIGS. 1-4. A method of use for StepNSoak Systems 100 may comprise the steps of: step one 501, opening one flexible non-permeable container 120; step two 502, inserting one appendage(s) 150; step three 503, pouring medicating fluid 160 into flexible non-permeable container 120; step four 504, wearing flexible non-permeable container 120 creating medicating action; step five 505, removing flexible non-permeable container 120; step six 506, cleaning flexible non-permeable container 120; and step seven 507, storing flexible non-permeable container 120 (for a future re-use.)

It should be noted that steps six 506 and step seven 507 are optional steps and may not be implemented in all cases. Optional steps of method of use 500 are illustrated using dotted lines in FIG. 5 so as to distinguish them from the other steps of method of use 500.

It should be noted that the steps described in the method of use can be carried out in many different orders according to user preference. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. An appendage soaking system comprising:
at least one flexible non-permeable container;
at least one securing assembly;
wherein at least one medicating solution is able to be suitably contained within said flexible non-permeable container;
wherein at least one said appendage is able to be inserted into said flexible non-permeable container; and
wherein said securing assembly is secured around said appendage thereby defining a confined inner volume wherein said medicating fluid is able to contact said appendage creating at least one medicating action.

2. The appendage soaking system of claim 1 wherein said at least one flexible non-permeable container comprises a plastic bag.

3. The appendage soaking system of claim 2 wherein said plastic bag comprises two seamed side edges and a seamed bottom edge and an open top.

4. The appendage soaking system of claim 2 wherein said plastic bag is comprised of heavy gauge material such that said flexible non-permeable container is reusable.

5. The appendage soaking system of claim 1 wherein said at least one securing assembly is comprised of at least one fused strap.

6. The appendage soaking system of claim 3 wherein said at least one fused strap is oriented parallel to said seamed bottom edge and said open top.

7. The appendage soaking system of claim 3 wherein said at least one fused strap is located in close proximity to said open top.

8. The appendage soaking system of claim 1 wherein said flexible non-permeable container comprises exactly two fused straps.

9. The appendage soaking system of claim 1 wherein said flexible non-permeable container comprises exactly one fused strap.

10. The appendage soaking system of claim 9 wherein said exactly one fused strap is fused in a middle of said fused strap to said flexible non-permeable container.

11. The appendage soaking system of claim 10 wherein said fused strap is made integral with said flexible non-permeable container.

12. The appendage soaking system of claim 11 wherein said fused strap is made integral with said flexible non-permeable container via a heated stamp.

13. The appendage soaking system of claim 1 wherein at least one said appendage comprises a hoof.

14. The appendage soaking system of claim 1 wherein at least one said appendage comprises a foot.

15. The appendage soaking system of claim 2 wherein said plastic bag comprises a medicating boot.

16. The appendage soaking system of claim 1 wherein said medicated fluid comprises a saline solution.

17. The appendage soaking system of claim 15 wherein said medicating boot is portable such that a user can freely move about while medicating said appendage.

18. An appendage soaking system comprising:
at least one flexible non-permeable container;
at least one securing assembly comprising at least one fused strap;
wherein said at least one flexible non-permeable container comprises a plastic bag including two seamed side edges and a seamed bottom edge and an openable top;
wherein said plastic bag is comprised of heavy gauge material such that said flexible non-permeable container is reusable;
wherein said at least one fused strap is oriented parallel to said seamed bottom edge and said open top and is located in close proximity to said openable top;
wherein said flexible non-permeable container comprises exactly one fused strap and is fused in the middle of said fused strap to said flexible non-permeable container and is thereby integral via a heat stamp with said flexible non-permeable container;
wherein said plastic bag comprises a medicating boot;
wherein at least one medicating solution is able to be suitably contained within said flexible container and comprises saline solution;
wherein at least one said appendage is a foot, hoof or paw and is able to be inserted into said flexible container;
wherein said securing assembly is secured around said appendage thereby defining a confined inner volume wherein said medicating fluid is able to contact said appendage creating at least one medicating action; and
wherein said medicating boot is portable such that a user can move about while medicating said appendage.

19. The appendage soaking system of claim 18 further comprising a kit including a plurality of said medicating boots; at least one said medicating fluid; and a set of user instructions.

20. A method for using an appendage soaking system comprising the steps of:
opening a flexible non-permeable container;
inserting an appendage;
pouring at least one medicated fluid into said flexible non-permeable container;
wearing said flexible non-permeable container creating at least one medicating action;
removing said flexible non-permeable container;
cleaning the flexible non-permeable container; and
storing the flexible non-permeable container for a future re-use.
